(19) United States
(12) Patent Application Publication
(10) Pub. No.: US 2026/0115376 A1
(43) Pub. Date: Apr. 30, 2026

(54) PORTABLE IV POLE ASSEMBLY

(71) Applicants: Sarah Scheerer, Lynchburg, VA (US); Devin Donnelly, Clayton, NC (US); Utsav Shah, Lake Ronkonkoma, NY (US); Douglas Argueta, Arlington, VA (US); Megan Gulian, McLean, VA (US)

(72) Inventors: Sarah Scheerer, Lynchburg, VA (US); Devin Donnelly, Clayton, NC (US); Utsav Shah, Lake Ronkonkoma, NY (US); Douglas Argueta, Arlington, VA (US); Megan Gulian, McLean, VA (US)

(21) Appl. No.: 19/361,137

(22) Filed: Oct. 17, 2025

Related U.S. Application Data
(60) Provisional application No. 63/711,335, filed on Oct. 24, 2024.

(51) Int. Cl. A61M 5/14

(52) U.S. Cl. CPC A61M 5/1417; A61M 5/1415

(57) ABSTRACT

A portable IV pole assembly for holding one or more IV bags and/or an IV pump includes a pole, with a telescoping portion in an upper portion of the pole. The portable IV pole assembly further includes a portable base, with a lower portion of the pole mounted to the portable base, and a first transverse member at the upper portion of the pole, A second transverse member is mounted to the telescoping portion of the pole, with each of the first and second transverse members having a plurality of IV bag supports, and with the IV bag supports of the second transverse member being offset horizontally from the IV bag supports of the first transverse member wherein the IV bag supports of the first transverse member and the second transverse member are accessible for supporting IV bags regardless of whether the second transverse member is raised or lowered.

### CROSS REFERENCE TO RELATED APPLICATION

The present application claims the benefit of U.S. provisional patent application Ser. No. 63/711,335, filed on Oct. 24, 2024, which is hereby incorporated herein by reference in its entirety.

### TECHNICAL FIELD AND BACKGROUND

The present disclosure relates to a portable IV pole assembly and, more particularly, to a portable IV pole assembly that can support one or more IV bags and/or pumps in an arrangement which can expand the number of IV bags and/or pumps supported by the portable IV pole assembly while increasing the stability and ergonomics of the IV pole assembly, and further enhancing the organization and visualization of the IV bags and IV pumps.

### SUMMARY OF THE INVENTION

In one form, a portable IV pole assembly for holding one or more IV bags and/or an IV pump includes a pole and a portable base. The pole has an upper portion, a lower portion, and a telescoping portion in the upper portion, with the lower portion of the pole mounted to the portable base. The portable IV pole assembly also includes first and second transverse members, with the first transverse member at the upper portion of the pole and the second transverse member mounted to the telescoping portion of the pole. Each of the first and second transverse members has a plurality of IV bag supports. The IV bag supports of the second transverse member are offset horizontally from the IV bag supports of the first transverse member wherein the IV bag supports of the first transverse member and the second transverse member are accessible for supporting IV bags regardless of whether the second transverse member is raised or lowered.

In one aspect, each of the IV bag supports of the first transverse member is formed from a W-shaped hook, which forms a pair of IV bag supports. Further, the pair of IV bag supports may be arranged in a plane, with each of the planes of the W-shaped hooks mounted at the first transverse member at a non-orthogonal angle to the central longitudinal axis of the first transverse member wherein IV bags can be supported by the IV bag supports of the first transverse member in staggered offset arrangements along the first transverse member.

In another aspect, each of the IV bag supports of the first and second transverse members is formed from a W-shaped hook, which provides a pair of IV bag supports. Further, each of the pair of IV bag supports may be arranged in a plane, with the planes of the W-shaped hooks mounted at the respective transverse member at non-orthogonal angles to the central longitudinal axis of the respective transverse member wherein IV bags can be supported by the IV bag supports of the transverse members in staggered offset arrangements along the respective transverse members.

For example, the planes of the W-shaped hooks may be mounted at the respective transverse member at an angle and a range of about 40 to 50°, and optionally about 45°.

In another form, a portable IV pole assembly includes a pole, a portable base, and a transverse member at the upper portion of the pole, with the lower portion of the pole mounted to the portable base. The transverse member is configured to support one or more IV bags. The pole further has a medial portion with a laterally offset from the upper portion and the lower portion of the pole. The medial portion is configured to receive IV equipment, such as an IV pump or pumps, and the lateral offset is sized to allow the equipment, such as the pump, to substantially align with the central axis of the pole when the IV equipment is mounted to the pole at the medial portion to improve stability and translational mobility of the portable IV pole assembly.

In one aspect, the medial portion of the pole is adjustable. For example, the medial portion may include an upper medial portion and a lower medial portion, with the upper medial portion being telescopingly mounted to the lower medial portion to allow adjustment to the vertical span of the medial portion.

In yet another form, a portable IV pole assembly for holding one or more IV bags includes a pole configured to support one or more IV bags and a portable base. The pole has an upper portion, a lower portion mounted to the portable base, and a central axis extending through the upper portion and the lower portion. The portable base has a plurality of arms extending radially outward relative to the central axis of the pole, with each arm having a bearing member for engaging a ground surface, with the bearing members defining a footprint of the IV pole assembly. The pole further has a medial portion being laterally offset from the upper portion and the lower portion, which forms a hand hold radially spaced outwardly relative to the central axis of the pole for a user of the portable IV pole assembly. The lateral offset is sized to allow a user to hold the pole of the IV pole while remaining outside of the footprint of the portable base with their arm bent at a reduced angle, for example at an orthogonal or acute angle, than when the pole has no laterally offset hand hold.

In one aspect, the medial portion is adjustable.

In a further aspect, the medial portion is vertically expandible. For example, the medial portion of the pole may include an upper medial portion and a lower medial portion, the upper medial portion being telescopingly mounted to the lower medial portion to allow vertical expansion of the medial portion.

In other aspects, the portable IV pole assembly further includes a second transverse member mounted to the pole. In a further aspect, the first and second transverse members each have a plurality of IV bag supports, with the IV bag supports of the second transverse member being offset horizontally from the IV bag supports of the first transverse member.

According to yet another form, a portable IV pole assembly for holding one or more IV bags includes a pole, a portable base, and a transverse member at the upper portion of the pole configured to support IV bags. The pole has an upper portion, a lower portion mounted to the portable base, and a central axis extending through the upper portion and the lower portion. The transverse member has central longitudinal axis and a plurality of IV bag supports, with each of the IV bag supports having a W-shaped hook forming a pair of IV bag supports arranged in a plane. The W-shaped hooks are mounted at the transverse member so that their planes are at non-orthogonal angles to the central longitudinal axis of the transverse member wherein IV bags can be supported by the IV bag supports in staggered offset arrangements along the transverse member.

In one aspect, the planes are at an angle in a range of about 40 to 50°, and optional about 45°.

In other aspect, the portable IV pole assembly further includes a second transverse member mounted to the pole, with each of the first and second transverse members having a plurality of IV bag supports, and with the IV bag supports of the second transverse member being offset horizontally from the IV bag supports of the first transverse member.

In yet another form, a portable IV pole assembly includes a pole configured to support one or more IV bags and a portable base. The pole has an upper portion, a lower portion, and a central axis extending through the upper portion and lower portion. The portable base has a central hub and a plurality of arms extending radially outward relative to the central hub, with the lower portion of the pole mounted to the central hub. Each arm has a proximal end mounted to the central hub and a distal end mounted to a caster wheel assembly for engagement with a ground surface, with at least one of the caster wheel assemblies being manually lockable. Each of the caster wheel assemblies is pivotally mounted to the distal end of a respective arm about a vertical swivel axis and has a caster wheel mounted about an axis of rotation. The caster wheels define a footprint of the IV pole assembly outside the distal ends of the arms.

In one aspect, the pole further includes a medial portion between the upper portion and the lower portion, which is laterally offset from the upper portion and the lower portion. The medial portion forms a hand hold radially spaced outwardly relative to the central axis for a user of the portable IV pole assembly.

### DETAILED DESCRIPTION OF THE DRAWINGS

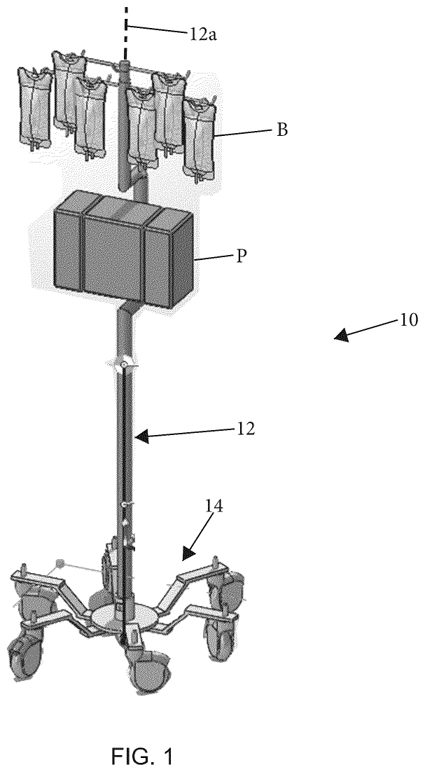

FIG. 1 is a perspective view of a portable IV pole assembly;

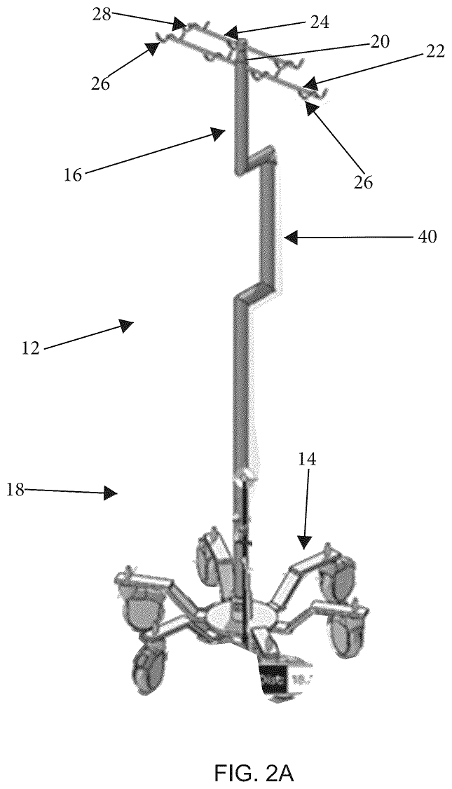

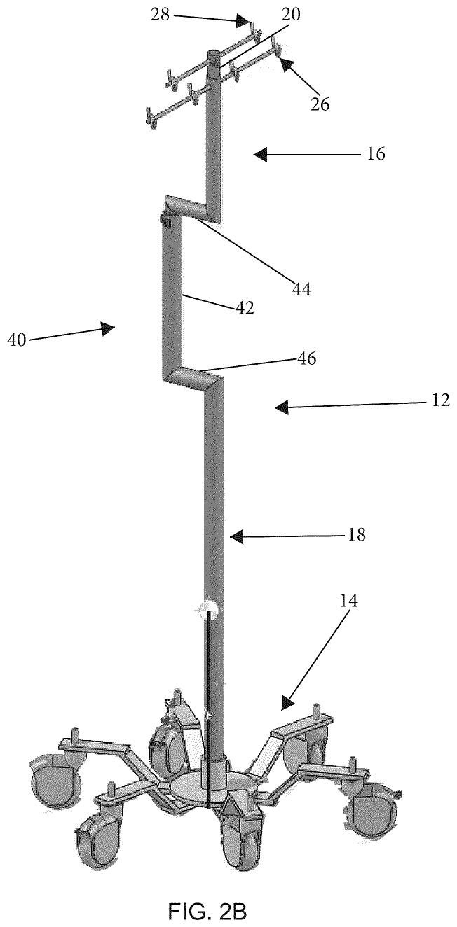

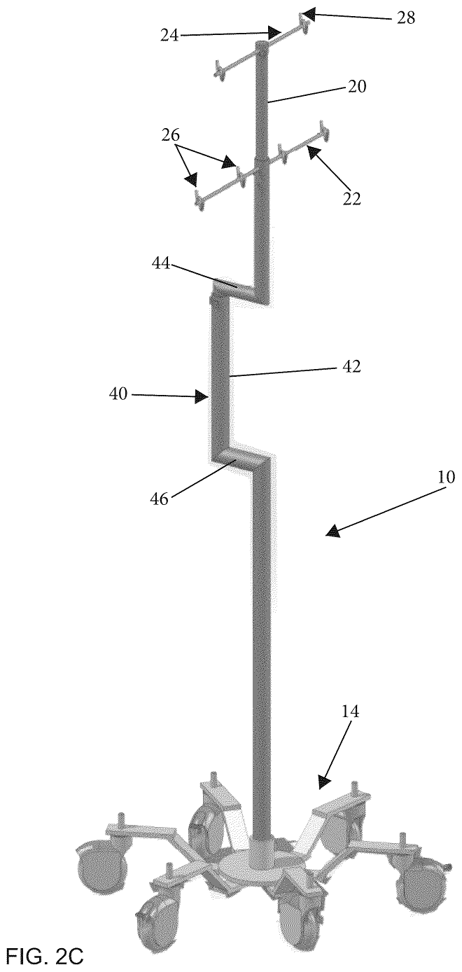

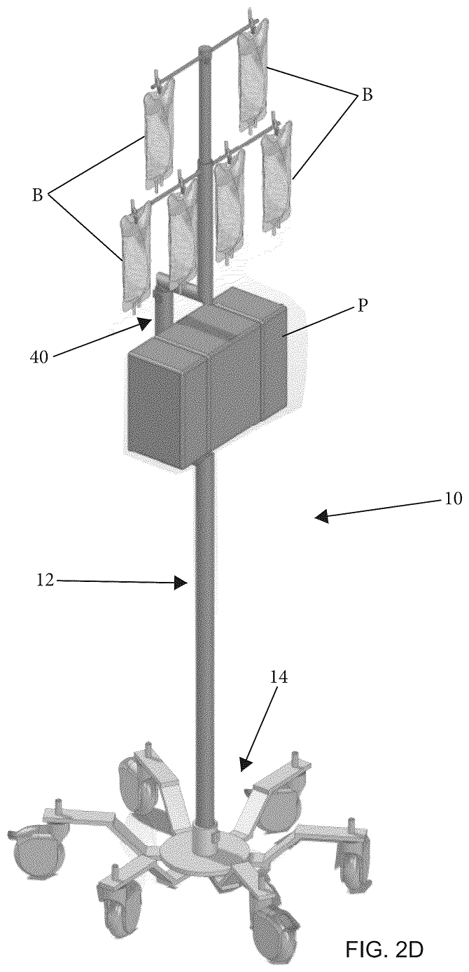

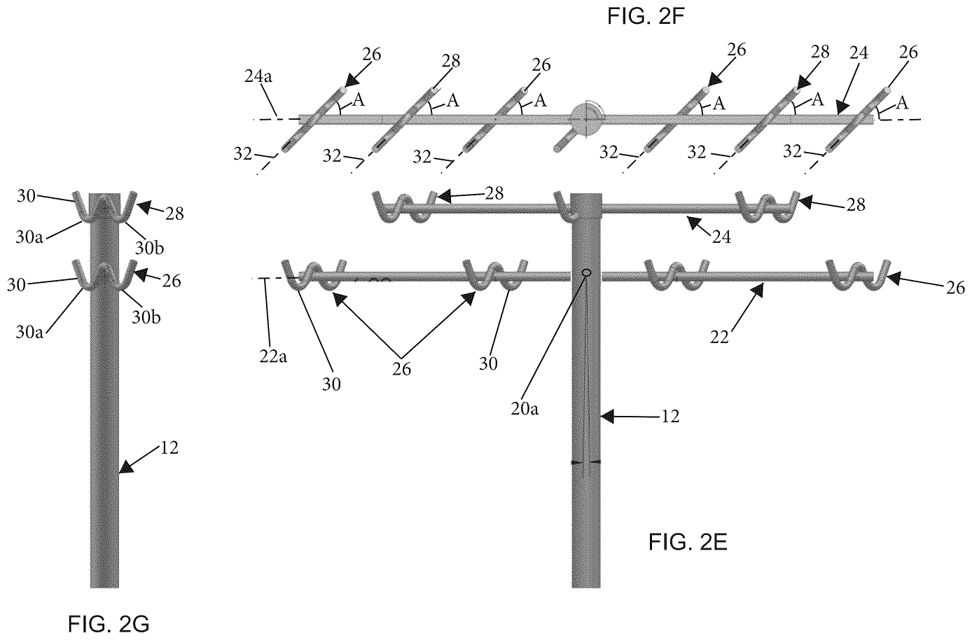

FIG. 2A is a similar view to FIG. 1 with the IV bags and IV pump removed for clarity;

FIG. 2B is another perspective view of the portable IV pole assembly;

FIG. 2C is a similar view to FIG. 2B illustrating the extension of the telescoping portion of the pole of the IV pole assembly;

FIG. 2D is a similar view to FIG. 2C illustrating the extension of the telescoping portion with IV bags and in IV pump mounted to the IV pole assembly;

FIG. 2E is an enlarged fragmentary view of the top portion of the pole of the portable IV pole assembly;

FIG. 2F is a top plan view of the pole of FIG. 2E;

FIG. 2G is an elevation view of one end of the pole of FIG. 2E;

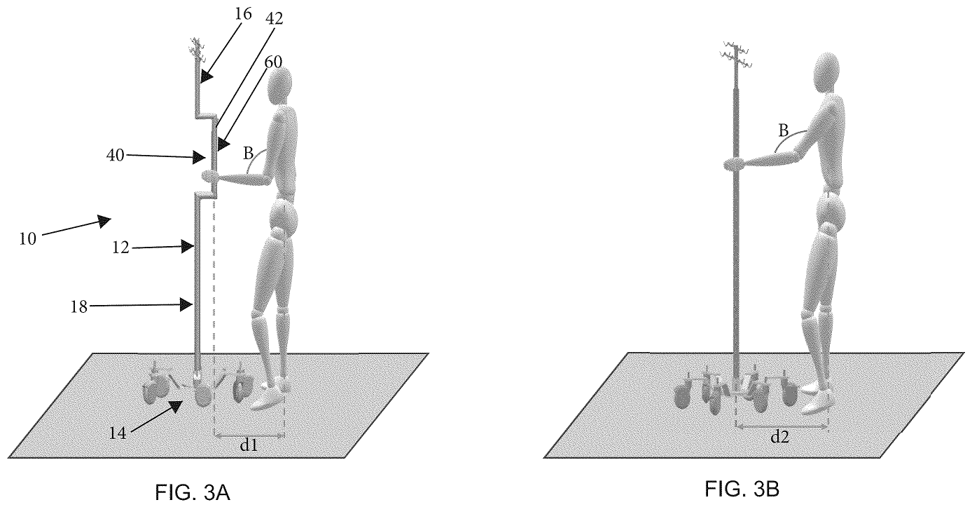

FIG. 3A is a perspective view of the portable IV pole assembly illustrating the use of the offset medial portion as a hand hold;

FIG. 3B is a perspective view of the use of a portable IV pole assembly without an offset medial portion;

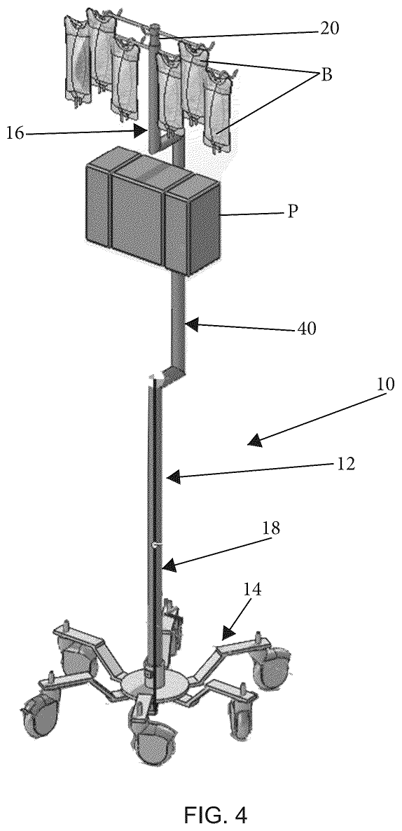

FIG. 4 is a perspective view of the portable IV pole assembly illustrating the extension of the medial portion of the pole;

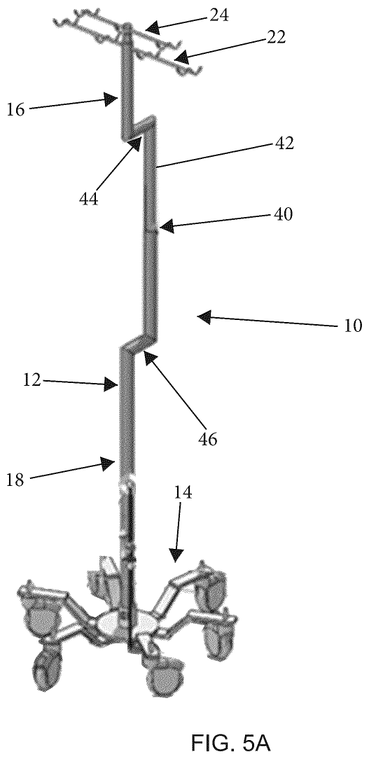

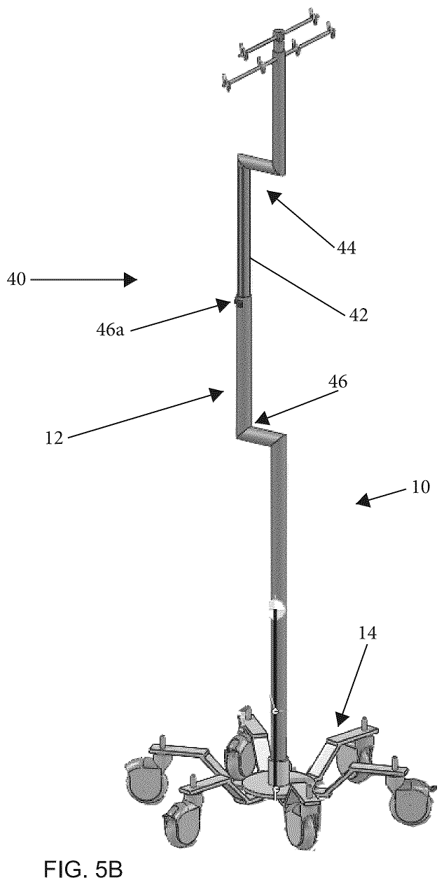

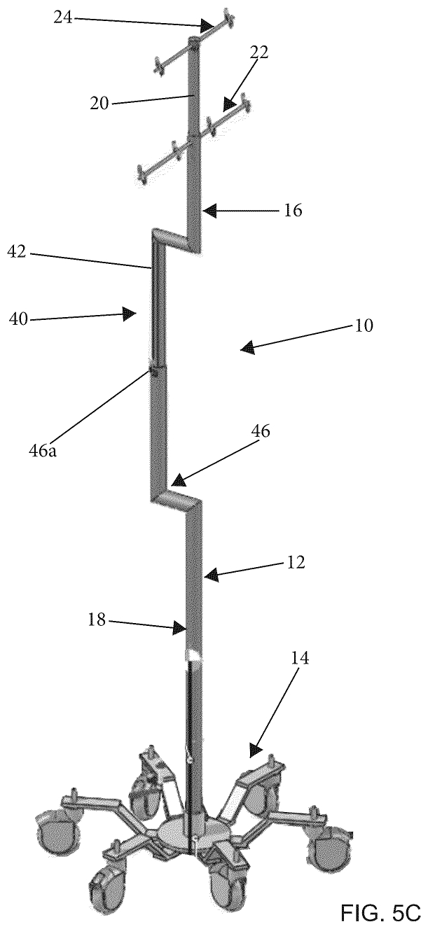

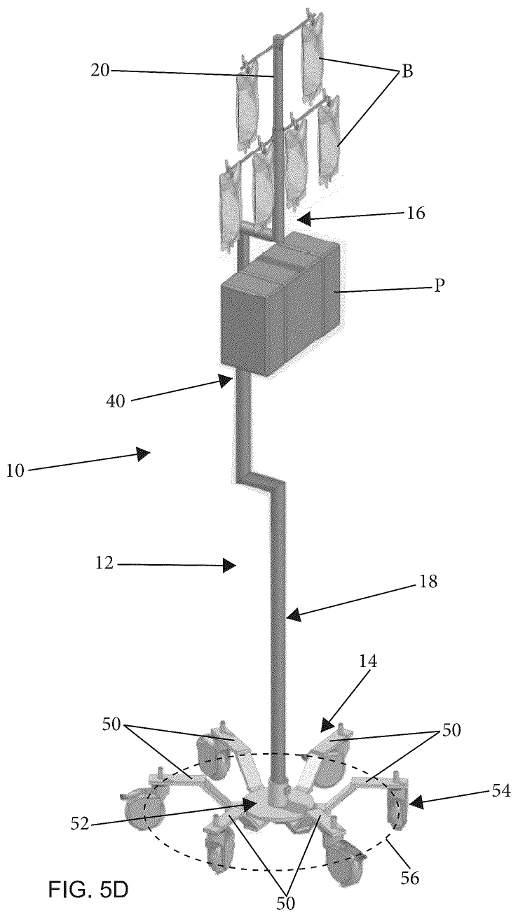

FIG. 5A is a perspective view of the portable IV pole assembly of FIG. 4 with the bags and IV pump removed for clarity:

FIG. 5B is another perspective view of the portable IV pole assembly with the bags and IV pump removed for clarity similar view to FIG. 5A;

FIG. 5C is a similar view to FIG. 5B with the telescoping portion of the pole illustrated in an extended configuration;

FIG. 5D is a similar view to FIG. 5C illustrated with IV bags and an IV pump mounted to the portable IV pole assembly;

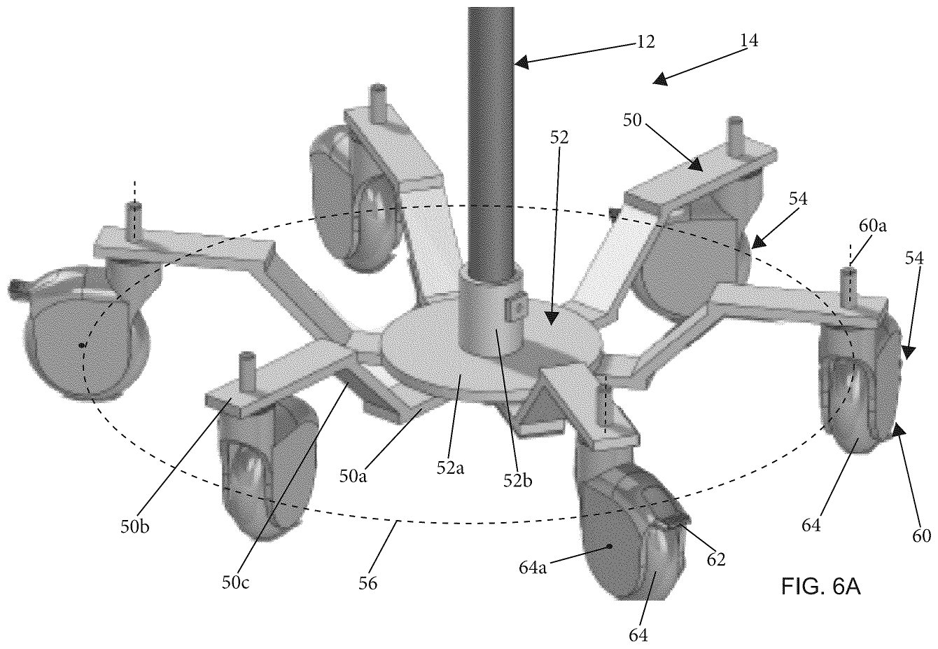

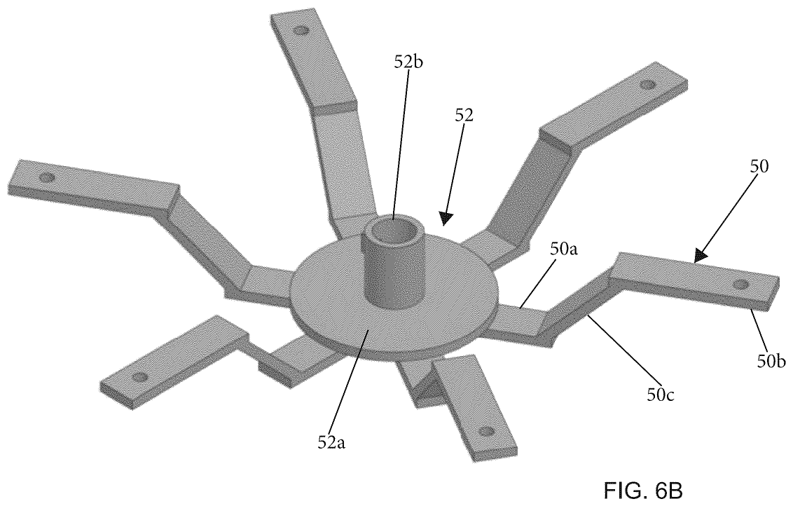

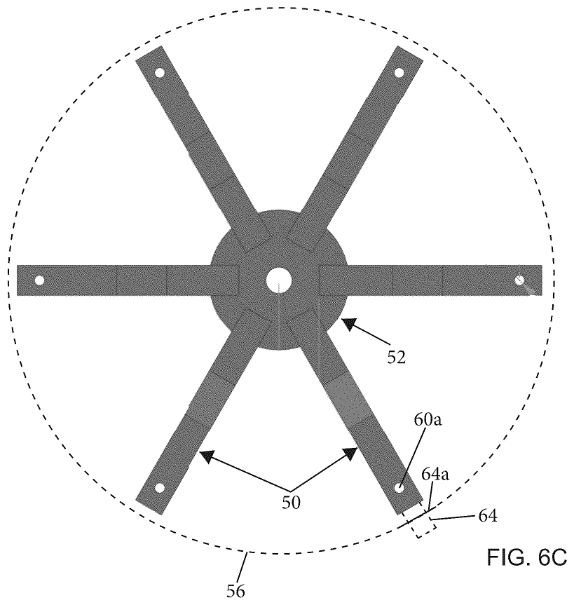

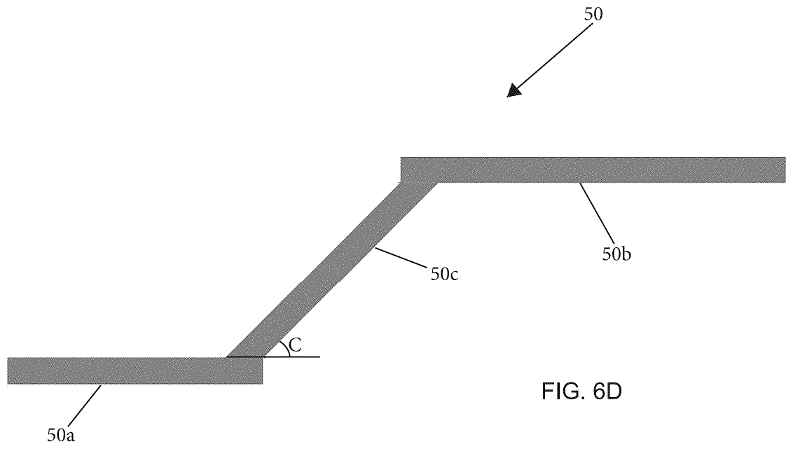

FIG. 6A is an enlarged perspective view of the portable base of the portable IV pole assembly;

FIG. 6B is a similar view to FIG. 6A with the bearing assemblies removed for clarity;

FIG. 6C is a bottom plan view of the portable base of the portable IV pole assembly with the bearing assemblies removed for clarity; and

FIG. 6D is an enlarged side elevation of one of the arms of the portable base of the portable IV pole assembly.

### DETAILED DESCRIPTION

Referring to FIG. 1, the numeral 10 generally designates a portable IV pole assembly for holding one or more IV bags and/or one or more IV pumps. As will be more fully described below, portable IV pole assembly 10 is configured to support one or more IV bags B and IV equipment, such as one or more IV pumps P, in more stable configurations and, further, that are more ergonomic so that portable IV pole assembly 10 does not interfere with the gate of a user, such as a caregiver, when the user moves the portable IV pole assembly 10 across a ground surface. Additionally, as will be described below, the IV bag supports may be configured and arranged to enhance the ability of a user to better organize the IV bags (and prevent stacking of the bags) so that their labels are more readily visible.

Referring again to FIG. 1, portable IV pole assembly 10 includes a pole 12 and a portable base 14. As will be more fully described below, pole 12 may be formed from several metal tube sections, such as stainless steel or aluminum tube sections, that are joined together by welding or, in some cases, in a telescoping arrangement to provide adjustment. As best seen in FIG. 2A, the pole 12 has an upper portion 16, a lower portion 18, and a telescoping portion 20 in the upper portion 16, with the lower portion 18 of the pole mounted to the portable base 14, such as by welding. Alternately, the lower portion may be removable mounted to the portable base so it can be mounted to a patient support apparatus, such as a hospital bed, for transport with the patient support apparatus and patient supported thereon.

The portable IV pole assembly 10 also include first and second transverse members 22, 24, with the first transverse member 22 mounted at the upper portion 16 of the pole 12 and the second transverse member 24 mounted to the telescoping portion 20 of the pole so that the height of the second transverse member 24 can be raised to a raised configuration (FIG. 2C) or lowered between the raised configuration and a fully lower configuration such as shown in FIG. 2A. Transverse members 22 and 24 may also be formed from metal rods, such as stainless steel or aluminum rods, that are mounted to their respective pole portions by welding.

To facilitate adjustment to the height of second transverse member 24, the upper portion 16 of the pole 12 may include a lock, such as a detent mechanism 20a (FIG. 2E), to releasably lock the height of the telescoping portion 20 (and hence the height of the second transverse member 24). Further, the pole 12 may also include an internal spring or gear mechanism to reduce the force needed to extend the telescoping portion 20. A suitable internal spring may include an internal hydraulic cylinder or an internal gas spring that may be mounted inside the pole and its extendible portion coupled to the telescoping portion, which can reduce the force required to raise or lower the telescoping portion 20 and the second transverse member 24. The gear mechanism may include a gear and track assembly, including with a motorized or spring biased gear, to reduce the force required to raise or lower the telescoping portion and the second transverse member 24.

Each of the first and second transverse members 22, 24 has a plurality of IV bag supports 26, 28 for supporting one or more IV bags B. As seen in FIGS. 1, 2A, 2B, 2C, and 2D, 2E, and 2F, the IV bag supports 28 of the second transverse member 24 are offset horizontally from the IV bag supports 26 of the first transverse member 22 wherein the IV bag supports 26, 28 of the first transverse member 22 and the second transverse member 24 are accessible for supporting IV bags regardless of whether the second transverse member 24 is raised or lowered.

In one aspect, each of the IV bag supports 26 of the first transverse member 22 is formed from a metal W-shaped hook 30, including a stainless steel or aluminum hook (e.g., cast from metal or formed from a metal rod). The W-shaped hook 30 is mounted to the first transverse member 22, for example, by welding, which forms a pair of IV bag supports 30a, 30b on opposed sides of the respective transverse member. Further, the pair of IV bag supports 30a, 30b may be arranged in a plane 32 (FIG. 2F), with the planes 32 of the W-shaped hooks 30 mounted at the first transverse member 22 at non-orthogonal angles A to the central longitudinal axis 22a of the first transverse member 22 wherein IV bags can be supported by the IV bag supports 26 on the first transverse member 22 in staggered offset arrangements along the first transverse member 22. Further, as noted, as a result, the labels of the bags may be more readily visible by a user.

In another aspect, each of the IV bag supports 26, 28 of the first and second transverse members 22, 24 is formed by a W-shaped hook 30, which may form a pair of IV bag supports 30a, 30b so that bags can be mounted on one side or both sides of the transverse members 22, 24. Further, as best seen in FIG. 2F, each of the pair of IV bag supports 30a, 30b of both transverse members 22, 24 may be arranged in a plane 32, with the planes 32 of the W-shaped hooks 30 mounted at the respective transverse member 22, 24 at non-orthogonal angles A to the central longitudinal axis 22a, 24a, of the respective transverse member 22, 24. In this manner, the IV bags B can be supported by the IV bag supports 26, 28 of the transverse members 22, 24 in staggered offset arrangements along the respective transverse members, again so the labels of the IV bags may be more visible to a user.

For example, the W-shaped hooks 30 may be mounted at the respective transverse member so that the planes 32 form an angle in a range of about 40 to 50°, and optionally about 45°, with respect to the respective central longitudinal axis 22a, 24a of the respective transverse members 22, 24.

In another form, as best seen in FIGS. 2A-2C and 5A, the pole 12 of portable IV pole assembly 10 may include a medial portion 40 that is laterally offset from the upper portion 16 and the lower portion 18 of the pole 12. The medial portion 40 is configured to receive an IV pump P (FIG. 2D) therein. The IV pump P can be directly mounted to the vertical section 42 of the medial portion 40 by conventional releasable fasteners, such as clamps (not shown), or indirectly mounted to the vertical section 42 of the medial portion 40 via a panel that is mounted to the vertical section 42 of the medial portion 40, with the pump or pumps P then mounted to the panel. The lateral offset is sized to substantially align the IV pump (or other IV equipment) with the central axis 12a (FIG. 1) of the pole when the IV pump (or other IV equipment) is mounted to the pole 12 in the medial portion to improve stability of the portable IV pole assembly. For example, the geometric center of the IV pump (or other IV equipment) or the center of gravity of the IV pump (or other IV equipment) may be substantially aligned with the central axis 12a (FIG. 1) of the pole 12 when the IV pump (or other IV equipment) is mounted to the pole 12.

Referring again to FIGS. 2B and 5A, the medial portion 40 of the pole may be adjustable. For example, the medial portion 40 may include an upper medial portion 44 and a lower medial portion 46, with the upper medial portion 44 being telescopingly mounted in the lower medial portion 46 to allow adjustment to the vertical span or height of the medial portion. For example, similar to telescoping portion 20, lower medial portion 46 may include a lock, such as a detent mechanism 46a (FIG. 5B), to releasably lock the height of the upper medial portion 44, and hence the length of vertical section 42 of the medial portion 40. In addition, pole 12 may also include an internal gear mechanism, such as a gear and track assembly, including a motorized or spring biased gear, to reduce the force required to raise or lower the upper medial portion 44. Alternately, an internal spring, such as an internal hydraulic cylinder or internal gas spring, may be used to reduce the force required to raise or lower the upper medial portion 44. In this manner the medial portion is vertically expandible to accommodate two or more pumps or other equipment.

As best seen in FIGS. 5D and 6A, the portable base 14 of portable IV pole assembly 10 has a plurality of arms 50 extending radially outward from a central hub 52 and relative to the central axis 12a of the pole 12. As will be more fully described below, the shape of each arm 50 may vary.

Each arm 50 has a bearing member 54, such as a caster wheel assembly, for engaging a ground surface. The bearing members define a footprint 56 of the IV pole assembly. As best seen in FIG. 3A, the vertical section 42 of medial portion 40, which is laterally offset from the upper portion and the lower portion, may form a hand hold 60 radially spaced outwardly relative to the central axis of the pole 12 for a user of the portable IV pole assembly. The lateral offset is sized to reduce the reach distance d1 to the pole 12 while a user is outside the footprint 56 of the IV pole assembly 10, This allows the user, such as a caregiver, to hold the pole with their arm bent at an angle B, for example at an orthogonal or acute angle, that is reduced from the angle that is required when the pole has no laterally offset hand hold (where the reach distance is d2 that is greater than d1) and where the angle of the arm is typically obtuse (such as shown in FIG. 3B). As a result, the medial portion forms an ergonomic configuration that can reduce interference by the portable IV pole assembly with the gait of the user.

As noted above, the medial portion may be adjustable so that the medial portion is vertically expandible. In addition to the accommodating two or more pumps (or other equipment), the vertically expandible medial portion may also increase the surface area of the hand hold to accommodate a greater range of user heights.

Referring again to FIG. 6A, as noted above, the portable base 14 has a central hub 52. Central hub 52 may be formed from a metal central plate 52a and a metal collar 52b, which is formed or otherwise attached, such as by welding, to central plate 52a. The lower end 18 of the pole 12 is received in the collar 52b and may be welded to the collar or may be secured therein by a fastener. Suitable metals include stainless steel or aluminum.

Arms 50 may also be formed from metal bars or plates. Arms 50 are formed or mounted, such as by welding, to the underside of plate 52a and have a stepped profile with their lower proximal ends 50a mounted or formed with central plate 52a and their upper distal ends 50b providing a mounting surface for the bearing assemblies, as described below. Arms 50 also include a sloped transition section 50c, which joins proximal and distal ends 50a, 50b together to form the stepped-shaped arms. Referring to FIG. 6D, the angle C formed between transition section 50c and distal end 50a may vary. For example, ancle C may range from about 40 to 75°, or about 68-69°, or about 45°.

As noted above, bearing assemblies 54 may comprise caster wheel assemblies 60, with optionally at least one of the caster wheel assemblies being manually lockable, for example, with a foot pedal 62. Each of the caster wheel assemblies 60 is pivotally mounted to the distal end 50b of a respective arm 50 about a vertical swivel axis 60a via a pivot pin and has a caster wheel 64 mounted about an axis of rotation 64a. The caster wheels 64 define the footprint 56 of the IV pole assembly 10, which when pivoted to their most extended position from the arms form a footprint 56 outside the distal ends of the arms.

Accordingly, the portable IV pole assembly described herein may support multiple IV bags and/or IV equipment, such as one or more IV pumps, in a stable and ergonomic arrangement that make the portable IV pole assembly more user friendly and easier to control.

While several forms of the IV pole assembly have been described herein other variations are also contemplated. Further, while not specifically described in each instance, the components of the IV pole may be formed from metal, such as stainless steel or aluminum or the like.

We claim:

1. A portable IV pole assembly for holding one or more IV bags and/or an IV pump, the IV pump having center of gravity, the portable IV pole assembly comprising:
a pole, the pole having an upper portion, a lower portion, and a telescoping portion in the upper portion;
a portable base, the lower portion of the pole mounted to the portable base;
a first transverse member at the upper portion of the pole; and
a second transverse member mounted to the telescoping portion of the pole, each of the first and second transverse members having a plurality of IV bag supports, the IV bag supports of the second transverse member being offset horizontally from the IV bag supports of the first transverse member wherein the IV bag supports of the first transverse member and the second transverse member are accessible for supporting IV bags regardless of whether the second transverse member is raised or lowered.

2. The portable IV pole assembly according to claim 1, wherein each of the IV bag supports of the first transverse member has a W-shaped hook forming a pair of IV bag supports.

3. The portable IV pole assembly according to claim 2, wherein the pair of IV bag supports are arranged in a plane, the first transverse member has a central longitudinal axis, and the planes of the W-shaped hooks being mounted at the first transverse member at non-orthogonal angles to the central longitudinal axis of the first transverse member wherein IV bags can be supported by the IV bag supports in offset arrangements along the first transverse member.

4. The portable IV pole assembly according to claim 3, wherein the planes of the W-shaped hooks are mounted at the transverse member at an angle and a range of about 40° to 50°.

5. The portable IV pole assembly according to claim 4, wherein the planes of the W-shaped hooks are mounted at the transverse member at an angle about 45°.

6. A portable IV pole assembly for holding one or more IV bags and/or an IV pump, the IV pump having center of gravity, the portable IV pole assembly comprising:
a pole;
a portable base, the pole having an upper portion, a lower portion mounted to the portable base, and a central axis extending through the upper portion and the lower portion; and
a transverse member at the upper portion of the pole configured for supporting one or more IV bags, the pole further having a medial portion having a laterally offset from the upper portion and the lower portion, the medial portion configured to receive an IV pump, and the lateral offset sized to substantially align the IV pump with the central axis of the pole when the IV pump is mounted to the pole at the medial portion to improve stability of the portable IV pole assembly.

7. The portable IV assembly according to claim 6, wherein the medial portion is adjustable.

8. The portable IV assembly according to claim 7, wherein the medial portion includes an upper medial portion and a lower medial portion, the upper medial portion being telescopingly mounted to the lower medial portion to provide adjustment to the medial portion.

9. The portable IV pole assembly according to claim 6, wherein the medial portion forms a hand hold radially spaced outwardly relative to the central axis for a user of the portable IV pole assembly, and the lateral offset being sized to allow a user to hold the pole of the IV pole while remaining outside of the footprint of the portable base with their arm bent at a reduced angle than when the pole has no laterally offset hand hold.

10. The portable IV pole assembly according to claim 9, wherein the medial portion is adjustable.

11. The portable IV assembly according to claim 10, wherein the medial portion is vertically expandible.

12. The portable IV assembly according to claim 10, wherein the medial portion includes an upper medial portion and a lower medial portion, the upper medial portion being telescopingly mounted to the lower medial portion to allow vertical expansion of the medial portion.

13. The portable IV assembly according to claim 10, wherein the transverse member comprises a first transverse member, further comprising a second transverse member mounted to the pole.

14. The portable IV assembly according to claim 13, wherein each of the first and second transverse members have a plurality of IV bag supports, the IV bag supports of the second transverse member being offset horizontally from the IV bag supports of the first transverse member.

15. A portable IV pole assembly for holding one or more IV bags, the portable IV pole assembly comprising:
a pole;
a portable base, the pole having an upper portion, a lower portion mounted to the portable base, and a central axis extending through the upper portion and the lower portion; and
a transverse member at the upper portion of the pole configured to support IV bags, the transverse member having a central longitudinal axis and a plurality of IV bag supports, each of the IV bag supports having a W-shaped hook forming a pair of IV bag supports arranged in a plane, and the planes of the W-shaped hooks mounted at the transverse member at non-orthogonal angles to the central longitudinal axis wherein IV bags can be supported by the IV bag supports in offset arrangements along the transverse member.

16. The portable IV pole assembly according to claim 15, wherein the planes of the W-shaped hooks are mounted at the transverse member at an angle and a range of about 40 to 50°.

17. The portable IV pole assembly according to claim 16, wherein the planes of the W-shaped hooks are mounted at the transverse member at an angle about 45°.

18. The portable IV pole assembly according to claim 15, wherein the transverse member comprises a first transverse member, further comprising a second transverse member mounted to the pole, each of the first and second transverse members having the plurality of IV bag supports, the IV bag supports of the second transverse member being offset horizontally from the IV bag supports of the first transverse member.

19. The portable IV pole assembly according to claim 15, wherein the pole further has a medial portion being laterally offset from the upper portion and the lower portion, and the medial portion forming a hand hold radially spaced outwardly relative to the central axis for a user of the portable IV pole assembly.

20. The portable IV pole assembly according to claim 6, wherein the portable base has a central hub and a plurality of arms extending radially outward relative to the central hub, the lower portion of the pole mounted to the portable base, each arm of the plurality of arms having a proximal end mounted to the central hub and a distal end mounted to a caster wheel assembly for engagement with a ground surface, each of the caster wheel assemblies being pivotally mounted to the distal end of a respective arm about a vertical swivel axis and having a caster wheel mounted about an axis of rotation, at least one of the caster wheel assemblies being manually lockable, and the caster wheels defining a footprint of the IV pole assembly outside the distal ends of the arms of the plurality of arms.
